(19) United States
(12) Patent Application Publication
(10) Pub. No.: US 2014/0248383 A1
(43) Pub. Date: Sep. 4, 2014

(54) SLEEP QUALITY IMPROVING AGENT

(71) Applicant: LION CORPORATION, Tokyo (JP)

(72) Inventors: Yoshihiro Urade, Ibaraki (JP); Toshio Gokita, Kanagawa (JP); Ikuko Kamada, Kanagawa (JP); Kiwamu Suzuki, Kanagawa (JP); Michiaki Murakoshi, Kanagawa (JP)

(73) Assignee: LION CORPORATION, Tokyo (JP)

(21) Appl. No.: 14/349,214

(22) PCT Filed: Oct. 9, 2012
(86) PCT No.: PCT/JP12/76088

§ 371 Date: Apr. 2, 2014

(30) Foreign Application Priority Data
Oct. 6, 2011 (JP) 2011-222255
Oct. 6, 2011 (JP) 2011-222256

(51) Int. Cl. A61K 36/63; A61K 31/353; A61K 36/15

(52) U.S. Cl. CPC A61K 36/63; A61K 36/15; A61K 31/353
USPC 424/770; 424/769; 514/456; 549/400

(57) ABSTRACT

An object is to provide a sleep quality improving agent that can sufficiently exert the effect of improving the quality of sleep and ensures safety for the body. A sleep quality improving agent containing, as an effective ingredient, a plant of the Fraxinus genus such as Fraxinus excelsior L., an extract thereof, Larix sibirica extract, dihydroquercetin, dihydrokaempferol, or naringenin, and a sleep quality improving composition containing the sleep quality improving agent are provided.

### TECHNICAL FIELD

The present invention relates to a sleep quality improving agent.

### BACKGROUND

Sleep is divided into two broad types: NREM sleep (non-rapid eye movement sleep) in which the cerebrum almost stops its activities and REM sleep (rapid eye movement sleep) in which, although the whole body is in a relaxed state, part of the brain is highly active and dreaming occurs. These two sleep types are repeated at regular intervals, forming sleep (FIG. 1). To achieve good sleep quality, the time from the onset of sleep until appearance of NREM sleep (sleep onset latency) must be short, and sufficient NREM sleep time and deep NREM sleep, particularly deep NREM sleep at the initial stage of sleep, must be ensured.

However, in recent years, as 24-hour society becomes more prevalent, the average sleep time of the Japanese decreases significantly, and this causes an increase in the number of patients with sleep complaints. A survey on awareness of health build-up in Heisei 8 (1996) reports that 21.4% of people have some sleep complaints. The survey also reports that the NREM sleep time of elderly people is significantly shorter than that of young people and the quality of sleep deteriorates with aging.

Examples of the sleep complaints may include difficulty in falling asleep, nightmares, sleepiness on awakening in the morning, no feeling of sufficient sleep, residual fatigue even after awakening in the morning, and sleepiness during daytime. These may lead to a reduction in work efficiency and unexpected accidents. The causes of these sleep complaints include not only a short sleep time but also lack of good quality sleep. More specifically, the causes are that the time from the onset of sleep until appearance of NREM sleep (sleep onset latency) is long, the length of NREM sleep is short, and deep NREM sleep is not obtained. To address these sleep complaints, it is contemplated to use medical sleeping pills. However, to use sleeping pills, a diagnosis by a physician is required. Sleeping pills have side effects such as an unpleasant awakening, dysmnesia, and dependence. As described above, sleeping pills are often difficult to use easily and safely. Some pharmaceutical products, such as benzodiazepine-based sleeping pills and barbiruturic acid-based sleeping pills, reduce the amount of NREM sleep, and the quality of sleep can rather deteriorates unless these are used appropriately.

Besides the pharmaceutical products, sleep improving agents produced from natural ingredients and food and drink ingredients are actively researched and developed, and various sleep improving agents have been proposed. Examples of the effective ingredients of the proposed sleep improving agents may include components derived from plants of the Withania genus belonging to the Solanaceae family (Patent Literature 1), fermented products of Hemerocallis fulva. var. sempervirens (Patent Literature 2), theamine derived from tea leaves (Patent Literature 3), and ginseng extracts (Non Patent Literature 1). However, the effects of these ingredients on the sleep onset latency and the length and depth of NREM sleep are not clear, and their effects of improving the quality of sleep are not sufficient.

Plants of the Fraxinus genus are dicotyledons belonging to the Oleaceae family that are distributed in the northern hemisphere. It has been reported that secoiridoids contained in an extract of the seeds of European ash, which is one of the plants of the Fraxinus genus, have physiological effects such as the effect of reducing blood pressure, the effect of reducing body weight, the effect of reducing body fat, and the effect of controlling insulin secretion and are effective for treatment of metabolic syndrome, type 2 diabetes, and hyperinsulinemia (Patent Literature 4).

### RELATED ART DOCUMENTS

### Patent Literature

Patent Literature 1: Japanese Patent Application Laid-Open No. 2006-28051
Patent Literature 2: Japanese Patent Application Laid-Open No. 2006-62998
Patent Literature 3: International Publication No. 2005/097101
Patent Literature 4: Japanese Translation of PCT International Application No. 2011-503009

### Non Patent Literature

Non Patent Literature 1: Young Ho Rhee, et al., Psychopharmacology, 101, p. 486-488 (1990)

### SUMMARY OF THE INVENTION

### Problem to be Solved by the Invention

However, it has not been known that plants of the Fraxinus genus and extracts thereof have the effect of improving the quality of sleep.

The present invention has been made in view of the above circumstances, and it is an object to provide a sleep quality improving agent that can exert a sufficient effect of improving the quality of sleep and ensures safety for the body.

### Means for Solving Problem

The present inventors have conducted repeated studies to achieve the above object. Consequently, the inventors have found that plants of the Fraxinus genus and extracts thereof have the effect of improving the quality of sleep. The inventors have found that Larix sibirica extract and its components, i.e., dihydroquercetin, dihydrokaempferol, and naringenin, have the effect of improving the quality of sleep. The present invention is based on the above findings.

[1] A sleep quality improving agent comprising, as an effective ingredient, one or two or more selected from the group consisting of a plant of the Fraxinus genus, an extract of the plant of the Fraxinus genus, Larix sibirica extract, dihydroquercetin, dihydrokaempferol, and naringenin.
[2] The sleep quality improving agent according to [1], wherein the effective ingredient is a plant of the Fraxinus genus or an extract thereof.
[3] The sleep quality improving agent according to [1] or [2], wherein the plant of the Fraxinus genus is Fraxinus excelsior L.
[4] The sleep quality improving agent according to [1] or [2], wherein the extract of the plant of the Fraxinus genus is Fraxinus excelsior L. extract.
[5] The sleep quality improving agent according to [4], wherein the Fraxinus excelsior L. extract is an extract of seeds of Fraxinus excelsior L.
[6] The sleep quality improving agent according to [1], wherein the effective ingredient is Larix sibirica extract.
[7] The sleep quality improving agent according to [1], wherein the effective ingredient is one or two or more compounds selected from the group consisting of dihydroquercetin, dihydrokaempferol, and naringenin.
[8] The sleep quality improving agent according to any one of [1] to [7] described above, wherein the sleep quality improving agent shortens sleep onset latency.
[9] The sleep quality improving agent according to any one of [1] to [8] described above, wherein the sleep quality improving agent deepens NREM sleep at an initial stage of sleep.
[10] The sleep quality improving agent according to any one of [1] to [9] described above, wherein the sleep quality improving agent increases a percentage of NREM sleep time in a total sleep time.
[11] The sleep quality improving agent according to any one of [1] to [10] described above, wherein the sleep quality improving agent improves less sleepiness feeling on awakening.
[12] The sleep quality improving agent according to any one of [1] to [11] described above, wherein the sleep quality improving agent improves ease of falling asleep and soundness of sleep.
[13] The sleep quality improving agent according to any one of [1] to [12] described above, wherein the sleep quality improving agent improves dreaming.
[14] The sleep quality improving agent according to any one of [1] to [13] described above, wherein the sleep quality improving agent improves feeling of fatigue.
[15] The sleep quality improving agent according to any one of [1] to [14] described above, wherein the sleep quality improving agent improves satisfaction with a length of sleep.
[16] A sleep quality improving composition containing the sleep quality improving agent according to any one of [1] to [15] described above.
[17] A method of use of a plant of the Fraxinus genus or an extract thereof to improve quality of sleep.
[18] A method of use of Larix sibirica extract to improve quality of sleep.
[19] A method of use of one or two or more compounds selected from the group consisting of dihydroquercetin, dihydrokaempferol, and naringenin.

Preferred embodiments of the present invention are as follows.

[1-1] A sleep quality improving agent containing, as an effective ingredient, a plant of the Fraxinus genus or an extract thereof.
[1-2] The sleep quality improving agent according to [1-1] described above, wherein the plant of the Fraxinus genus is Fraxinus excelsior L.
[1-3] The sleep quality improving agent according to [1-1] described above, wherein the extract of the plant of the Fraxinus genus is Fraxinus excelsior L. extract.
[1-4] The sleep quality improving agent according to any one of [1-1] to [1-3] described above, wherein the sleep quality improving agent shortens sleep onset latency.
[1-5] The sleep quality improving agent according to any one of [1-1] to [1-4] described above, wherein the sleep quality improving agent deepens NREM sleep at an initial stage of sleep.
[1-6] The sleep quality improving agent according to any one of [1-1] to [1-5] described above, wherein the sleep quality improving agent increases a percentage of NREM sleep time in a total sleep time.
[1-7] The sleep quality improving agent according to any one of [1-1] to [1-6] described above, wherein the sleep quality improving agent improves less sleepiness feeling on awakening.
[1-8] The sleep quality improving agent according to any one of [1-1] to [1-7] described above, wherein the sleep quality improving agent improves ease of falling asleep and soundness of sleep.
[1-9] The sleep quality improving agent according to any one of [1-1] to [1-8] described above, wherein the sleep quality improving agent improves dreaming.
[1-10] The sleep quality improving agent according to any one of [1-1] to [1-9] described above, wherein the sleep quality improving agent improves feeling of fatigue.
[1-11] The sleep quality improving agent according to any one of [1-1] to [1-10] described above, wherein the sleep quality improving agent improves satisfaction with a length of sleep.
[1-12] The sleep quality improving agent according to any one of [1-1] to [1-11] described above, wherein the Fraxinus excelsior L. extract is an extract of seeds of Fraxinus excelsior L.
[1-13] A sleep quality improving composition containing the sleep quality improving agent according to any one of [1-1] to [1-12] described above.
[1-14] A method of use of a plant of the Fraxinus genus or an extract thereof to improve quality of sleep.

Other preferred embodiments of the present invention are as follows.

[2-1] A sleep quality improving agent containing, as an effective ingredient, Larix sibirica extract.
[2-2] A sleep quality improving agent containing, as an effective ingredient, one or two or more compounds selected from the group consisting of dihydroquercetin, dihydrokaempferol, and naringenin.
[2-3] The sleep quality improving agent according to any one of [2-1] to [2-2] described above, wherein the sleep quality improving agent improves less sleepiness feeling on awakening.
[2-4] The sleep quality improving agent according to any one of [2-1] to [2-3] described above, wherein the sleep quality improving agent improves ease of falling asleep and soundness of sleep.
[2-5] The sleep quality improving agent according to any one of [2-1] to [2-4] described above, wherein the sleep quality improving agent improves dreaming.
[2-6] The sleep quality improving agent according to any one of [2-1] to [2-5] described above, wherein the sleep quality improving agent improves feeling of fatigue.
[2-7] The sleep quality improving agent according to any one of [2-1] to [2-6] described above, wherein the sleep quality improving agent improves satisfaction with a length of sleep.
[2-8] A sleep quality improving composition containing the sleep quality improving agent according to any one of [2-1] to [2-7] described above.
[2-9] A method of use of Larix sibirica extract to improve quality of sleep.
[2-10] A method of use of one or two or more compounds selected from the group consisting of dihydroquercetin, dihydrokaempferol, and naringenin to improve quality of sleep.

### Effect of the Invention

The present invention provides a sleep quality improving agent that can exert the effect of improving the quality of sleep sufficiently and is useful as food products, pharmaceutical products, and quasi-pharmaceutical products. The sleep quality improving agent of the present invention comprises, as an effective ingredient, one or two or more compounds selected from the group consisting of plants of the Fraxinus genus, extracts of the plants of the Fraxinus genus, Larix sibirica extract, and components of Larix sibirica extract, i.e., dihydroquercetin, dihydrokaempferol, and naringenin, so that safety for the body is ensured.

### BRIEF DESCRIPTION OF DRAWINGS

FIG. 1 is a diagram illustrating a general sleep pattern.

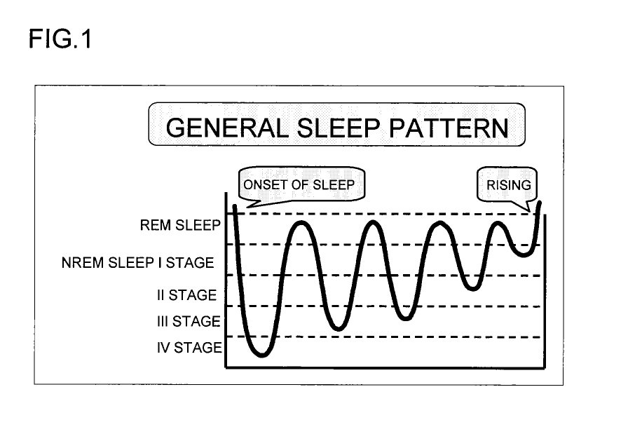

FIG. 2 is a graph showing the cumulative amount of activity of mice 8.5 hours after sample administration in Example 1-1 and Comparative Example 1-1.

FIG. 3 is a graph showing the percentage of each of sleep stages (wakefulness or NREM sleep) in Example 1-2 and Comparative Example 1-2.

FIG. 4 is a graph showing the percent change in sleep onset latency in Example 1-3 relative to that in Comparative Example 1-3.

FIG. 5 is a graph showing the percent change in the power value of delta waves at the initial stage of sleep in Example 1-3 relative to that in Comparative Example 1-3.

FIG. 6 is a graph showing the percent change in the score of factor I in Example 1-3 relative to that in Comparative Example 1-3.

FIG. 7 is a graph showing the percent change in the score of factor II in Example 1-3 relative to that in Comparative Example 1-3.

FIG. 8 is a graph showing the percent change in the score of factor III in Example 1-3 relative to that in Comparative Example 1-3.

FIG. 9 is a graph showing the percent change in the score of factor IV in Example 1-3 relative to that in Comparative Example 1-3.

FIG. 10 is a graph showing the percent change in the score of factor V in Example 1-3 relative to that in Comparative Example 1-3.

FIG. 11 is a graph showing the percent change in the score of feeling of fatigue in Example 1-3 relative to that in Comparative Example 1-3.

FIG. 12 is a graph showing the cumulative amount of activity of mice 6 hours after sample administration for each of Example 2-1 and Comparative Example 2-1.

FIG. 13 is a graph showing the cumulative amount of activity of mice 6 hours after sample administration for each of Example 2-2 and Comparative Example 2-2.

FIG. 14 is a graph showing the percent change in the score of factor I in Example 2-3 relative to that in Comparative Example 2-3.

FIG. 15 is a graph showing the percent change in the score of factor II in Example 2-3 relative to that in Comparative Example 2-3.

FIG. 16 is a graph showing the percent change in the score of factor III in Example 2-3 relative to that in Comparative Example 2-3.

FIG. 17 is a graph showing the percent change in the score of factor IV in Example 2-3 relative to that in Comparative Example 2-3.

FIG. 18 is a graph showing the percent change in the score of factor V in Example 2-3 relative to that in Comparative Example 2-3.

### EMBODIMENTS FOR CARRYING OUT THE INVENTION

The sleep quality improving agent of the present invention comprises, as an effective ingredient, one or two or more selected from the group consisting of plants of the Fraxinus genus, extracts of the plants of the Fraxinus genus, Larix sibirica extract, dihydroquercetin, dihydrokaempferol, and naringenin.

A first embodiment of the present invention is a sleep quality improving agent containing a plant of the Fraxinus genus or an extract of the plant of the Fraxinus genus as an effective ingredient.

A second embodiment of the present invention is a sleep quality improving agent containing Larix sibirica extract as an effective ingredient. A third embodiment of the present invention is a sleep quality improving agent containing, as an effective ingredient, one or two or more compounds selected from the group consisting of dihydroquercetin, dihydrokaempferol, and naringenin.

A plant of the Fraxinus genus means part or the whole body of the plant of the Fraxinus genus, and the extract of the plant of the Fraxinus genus means an extract extracted from the plant of the Fraxinus genus. In the present invention, any of plants of the Fraxinus genus and extracts of the plants of the Fraxinus genus or a combination thereof may be used as the effective ingredient. From the viewpoint of ease of administration and ease of formulation into a preparation, it is preferable to use an extract of a plant of the Fraxinus genus as the effective ingredient.

The plants of the Fraxinus genus are dicotyledons belonging to the Oleaceae family. The plants of the Fraxinus genus include deciduous plants, evergreen plants, trees, and bushes, and any of them can be used in the present invention. The plants of the Fraxinus genus are distributed, for example, in the northern hemisphere (for example, Europe, East Asia, West Asia, North America, and Mediterranean). Plants distributed in other regions may be used. The plant used may be either a naturally grown plant or an artificially grown plant. Examples of the plants of the Fraxinus genus may include F. americana L. (trivial name: White Ash), F. chinensis Roxb. (trivial name: Chinese Ash), F. excelsior L. (trivial name: Ash, Common Ash or European Ash), F. griffithii C. B. Clarke (trivial name: Griffith's Ash), F. japonica Bl. (trivial name: Japanese Ash), F. bungeana DC., F. lanuginosa, F. mandshurica Rupr. var. japonica Maxim., F. ornus L. (trivial name: Manna Ash), F. spaethiana Lingelsh (F. platypoda Oliv), F. sieboldiana Blume (trivial name: Japanese Flowering Ash), F. nigra Marshall (trivial name: Black Ash), and F. paxiana Lingelsh. Of these, Fraxinus excelsior L. is preferred. Fraxinus excelsior L. is a deciduous tree with lateral inflorescences. Fraxinus excelsior L. is distributed mainly in ranges from Europe to West Asia. Fraxinus excelsior L. distributed in other regions may be used. The Fraxinus excelsior L. used may be either a naturally grown plant or an artificially grown plant.

The plant of the Fraxinus genus used as the effective ingredient in the present invention may be part or the whole body of the plant of the Fraxinus genus, and seeds of the plant of the Fraxinus genus are preferred. Part or the whole body of the plant may be used without any processing or may be subjected to processing such as concentration, drying, pulverization, etc. In terms of ease of administration and ease of formulation into a preparation, dried and pulverized powder is preferred. No particular limitation is imposed on the form of the plant of the Fraxinus genus, and the plant may be in a powder or paste form. One type of plant of the Fraxinus genus may be used. Alternatively, a combination of two or more types of plants may be used, or a combination of two or more different portions of a plant may be used.

The extract of the plant of the Fraxinus genus can be extracted from (part or the whole body of) the plant of the Fraxinus genus using an extractant. No particular limitation is imposed on the portion of the plant of the Fraxinus genus that is used for extraction. Such a portion may by the whole body of the plant or part of the plant such as leaves, bark, xylem, seeds or flowers and is preferably seeds. The extractant used may be water, an organic solvent (for example, one type of solvent selected from alcohols, acetone, etc., or a mixture of two or more types of such solvents), or a mixture of water and an organic solvent. Of these, water, a mixture of water and an alcohol, and an alcohol are preferred. The alcohol is preferably ethanol or methanol. The time and temperature of extraction may be appropriately determined according to the portion of the plant of the Fraxinus genus that is used for extraction, the type of solvent, etc. The time of extraction is preferably about 2 hours to about 24 hours. The temperature of extraction is preferably 20° C. to 100° C. and more preferably 50° C. to 70° C. The extract of the plant of the Fraxinus genus may be a crude extract extracted from the plant of the Fraxinus genus or may be a product obtained by subjecting the crude extract to processing such as concentration, drying, pulverization, etc. An extract from which impurities have been removed by treatment using a partition method or purification treatment (for example, after adsorption treatment using an ion-exchange resin, columns, etc., elution with a solvent is performed, and then concentration treatment is performed as needed) may be used. No particular limitation is imposed on the form of the extract of the plant of the Fraxinus genus, and the extract may be in a powder or paste form. One type of extract of the plant of the Fraxinus genus may be used, or a combination of two or more types of extracts extracted, for example, under different extraction conditions may be used.

The plant of the Fraxinus genus or its extract used as the effective ingredient in the present invention is preferably the seeds of Fraxinus excelsior L. or an extract thereof. The extract of the seeds of Fraxinus excelsior L. is preferred.

Extracts of plants of the Fraxinus genus are commercially available, and such a commercial product may be used. Examples of the commercial product of the extract of Fraxinus excelsior L. may include “fraxipure (trade name)” (NATUREX).

Larix sibirica is a coniferous plant belonging to the Pinaceae family. Larix sibirica is distributed in Siberia and the Far East. Larix sibirica distributed in other regions may be used. Larix sibirica may be either naturally grown plant or artificially grown plant.

The Larix sibirica extract means an extract extracted from part or the whole body of Larix sibirica. The Larix sibirica extract can be extracted from part or the whole body of Larix sibirica using an extractant. No particular limitation is imposed on the portion of Larix sibirica that is used for extraction. Such a portion may by the whole body of the plant or part of the plant such as leaves, bark, xylem, seeds or flowers and is preferably bark or xylem and more preferably xylem. The extractant used may be water, an organic solvent (for example, one type of solvent selected from alcohols, acetone, etc., or a mixture of two or more types of such solvents), or a mixture of water and an organic solvent. Of these, water, a mixture of water and an alcohol, and an alcohol are preferred. The alcohol is preferably ethanol or methanol. The time and temperature of extraction may be appropriately determined according to the portion of Larix sibirica that is used for extraction, the type of solvent, etc. The Larix sibirica extract may be a crude extract extracted from Larix sibirica or may be a product obtained by subjecting the crude extract to processing such as concentration, drying, pulverization, etc. An extract from which impurities have been removed by treatment using a partition method or purification treatment (for example, after adsorption treatment using an ion-exchange resin, columns, etc., elution with a solvent is performed, and then concentration treatment is performed as needed) may be used. A product obtained by finely pulverizing Larix sibirica, wetting the pulverized product with water vapor, subjecting the resultant product to extraction with acetone, and then purifying the resultant product as described in Russian Patent No. 2091076 may be used as the Larix sibirica extract. No particular limitation is imposed on the form of the Larix sibirica extract, and the Larix sibirica extract may be in a powder or paste form. One type of Larix sibirica extract may be used, or a combination of two or more types of extracts extracted, for example, under different extraction conditions may be used.

Larix sibirica extract is commercially available, and a commercial product may be used. Examples of the commercial product of the Larix sibirica extract may include “DIKVERTIN (trade name)” (FLAVIR, Irkutsk, Russia).

Each of dihydroquercetin, dihydrokaempferol, and naringenin is a component of the Larix sibirica extract and is a polyphenol having a flavan skeleton. The content of dihydroquercetin in Larix sibirica is largest, and the contents of dihydrokaempferol and naringenin are small. The effective ingredient in the present invention may be any of dihydroquercetin, dihydrokaempferol, and naringenin or a combination of two or more thereof. When the effective ingredient in the present invention is one or two or more compounds selected from dihydroquercetin, dihydrokaempferol, and naringenin, the effective ingredient is preferably dihydroquercetin alone or a combination including dihydroquercetin, and more preferably dihydroquercetin alone.

No particular limitation is imposed on the method of producing dihydroquercetin, dihydrokaempferol, and naringenin. These may be artificial products produced by, for example, chemical synthesis or products extracted from Larix sibirica or Larix sibirica extract and purified.

No particular limitation is imposed on the dosage of the sleep quality improving agent of the present invention so long as the effects of the invention are not impaired. The dosage may be appropriately changed according to factors such as the age and conditions of a living body to which the agent is administered. A preferred dosage of the sleep quality improving agent for obtaining the intended effect is as follows.

When the effective ingredient is Fraxinus excelsior L., the dosage of Fraxinus excelsior L. per day is generally 0.045 g to 18 g, preferably 0.2 g to 13.5 g, and more preferably 0.45 g to 9 g. When the effective ingredient is Fraxinus excelsior L. extract, the dosage of Fraxinus excelsior L. extract per day is generally 10 mg to 4,000 mg, preferably 50 mg to 3,000 mg, and more preferably 100 mg to 2,000 mg.

When the effective ingredient is Larix sibirica extract, the dosage of Larix sibirica extract per day is generally 3 mg to 1,000 mg, preferably 15 mg to 500 mg, more preferably 20 mg to 200 mg, and still more preferably 30 mg to 120 mg. When the effective ingredient is dihydroquercetin, the dosage of dihydroquercetin per day is generally 2.7 mg to 900 mg, preferably 13.5 mg to 450 mg, more preferably 18 mg to 180 mg, and still more preferably 27 mg to 108 mg. When the effective ingredient is dihydrokaempferol, the dosage of dihydrokaempferol per day is generally 2.7 mg to 900 mg, preferably 13.5 mg to 450 mg, more preferably 18 mg to 180 mg, and still more preferably 27 mg to 108 mg. When the effective ingredient is naringenin, the dosage of naringenin per day is generally 2.7 mg to 900 mg, preferably 13.5 mg to 450 mg, more preferably 18 mg to 180 mg, and still more preferably 27 mg to 108 mg.

The sleep quality improving agent of the present invention in its original form may be formulated into a preparation and used as a final product such as a food or drink product, a pharmaceutical product, or a quasi-pharmaceutical product. In addition, the sleep quality improving agent can be used as additives for food and drink products, additives for pharmaceutical products, and additives for quasi-pharmaceutical products. In this manner, the effect of improving the quality of sleep can be imparted to food and drink products, pharmaceutical products, and quasi-pharmaceutical products.

It is sufficient that the sleep quality improving agent of the present invention comprises, as the effective ingredient, one or two or more selected from the group consisting of plants of the Fraxinus genus, extracts of plants of the Fraxinus genus, Larix sibirica extract, dihydroquercetin, dihydrokaempferol, and naringenin. The sleep quality improving agent may comprise additional components other than the above components. Examples of the additional components may include components for ensuring stability mainly during storage and distribution such as a storage stabilizer. One or two or more types (preferably approximately one to three types and more preferably approximately one type) of components selected from the components constituting a target final product such as a food or drink product, a pharmaceutical product, or a quasi-pharmaceutical product may be added in advance to the sleep quality improving agent.

The sleep quality improving agent of the present invention may be combined with a component other than plants of the Fraxinus genus, extracts of plants of the Fraxinus genus, Larix sibirica extract, dihydroquercetin, dihydrokaempferol, and naringenin to prepare a sleep quality improving composition.

No particular limitation is imposed on the component, other than plants of the Fraxinus genus, extracts of plants of the Fraxinus genus, Larix sibirica extract, dihydroquercetin, dihydrokaempferol, and naringenin, contained in the sleep quality improving composition of the present invention, so long as the object of the invention is not impaired. Examples of such a component may include pharmaceutically acceptable additives such as excipients, disintegrators, binding agents, lubricants, coating agents, coloring agents, color formers, taste masking agents, flavoring agents, antioxidants, antiseptics, taste agents, acidulants, sweeteners, fortifiers, vitamin compounds, inflating agents, thickeners, and surfactants. One or two or more types of additives that do not impair the effect of improving the quality of sleep and the characteristics necessary for the formulation such as the stability of the formulation and are appropriate for the dosage form of the final product may be selected from the above additives. The above component may have the effect of improving the quality of sleep. The sleep quality improving composition of the present invention may comprise one type of component other than plants of the Fraxinus genus, extracts of plants of the Fraxinus genus, Larix sibirica extract, dihydroquercetin, dihydrokaempferol, and naringenin or a combination of two or more types of such components.

The dosage of the sleep quality improving composition of the present invention may be controlled such that the amount of a plant of the Fraxinus genus, an extract of a plant of the Fraxinus genus, Larix sibirica extract, dihydroquercetin, dihydrokaempferol, or naringenin is within the range described above for the dosage of the sleep quality improving agent of the present invention.

The sleep quality improving composition of the present invention in its original form may be used as a final product such as a food or drink product, a pharmaceutical product, or a quasi-pharmaceutical product. The composition may also be used as additives for food and drink products, additives for pharmaceutical products, and additives for quasi-pharmaceutical products. In this manner, the effect of improving the quality of sleep can be imparted to food and drink products, pharmaceutical products, and quasi-pharmaceutical products.

No particular limitation is imposed on the form of administration of the sleep quality improving agent of the present invention and the sleep quality improving composition of the present invention. Examples of the form of administration may include oral administration such as buccal administration and sublingual administration and parenteral administration such as intravenous administration, intramuscular administration, subcutaneous administration, percutaneous administration, transnasal administration, and pulmonary administration. Of these, less invasive administration forms are preferred, and oral administration is more preferred. Furthermore, oral administration as a food or drink product is more preferred.

No particular limitation is imposed on the dosage forms of the sleep quality improving agent of the present invention and the sleep quality improving composition of the present invention. Examples of the dosage form for oral administration may include a liquid form (liquid agents), a syrup form (syrup agents), a solid form (tablets), a capsule form (capsules), a powdery form (granules and fine particles), a soft capsule form (soft capsules), a semi-liquid form, a cream form, and a paste form. Examples of the dosage form for parenteral administration may include liquid agents such as injections and nasal drops and an atomized form such as sprays and inhalants.

No particular limitation is imposed on the time of administration of the sleep quality improving agent of the present invention and the sleep quality improving composition of the present invention. These are administered generally before going to bed, preferably between three hours before going to bed and the time of going to bed, more preferably between two hours before going to bed and the time of going to bed, still more preferably between one hour before going to bed and the time of going to bed, and particularly preferably one hour before going to bed.

The effective ingredient of the sleep quality improving agent of the present invention has a significant effect of improving the quality of sleep.

The “quality of sleep” in the present invention means that sleep gives the tired body and brain a rest. Even when the sleep time is long, fatigue cannot be sufficiently relieved if the quality of sleep is low. Examples of the index for measuring the quality of sleep may include the time from the onset of sleep until appearance of NREM sleep (hereinafter referred to as sleep onset latency), the depth of NREM sleep at the initial stage of sleep, the percentage of NREM sleep time in the total sleep time, less sleepiness feeling on awakening, ease of falling asleep and soundness of sleep, dreaming, satisfaction with the length of sleep, and feeling of fatigue. Of these, the sleep onset latency, the depth of NREM sleep at the initial stage of sleep, the percentage of NREM sleep time in the total sleep time, less sleepiness feeling on awakening, ease of falling asleep and soundness of sleep, dreaming, satisfaction with the length of sleep, and the feeling of fatigue are preferred. The NREM sleep at the initial stage of sleep means NREM sleep that appears immediately after the onset of sleep and before appearance of REM sleep. The sleep onset latency can be checked using an record of brain waves obtained by measuring brain waves during sleep, as will be shown in Examples. The depth of NREM sleep at the initial stage of sleep can be checked using the power value of delta waves in the brain waves during sleep, as will be shown in Examples. As the power value of delta waves increases, the depth of sleep increases. The percentage of NREM sleep time in the total sleep time can also be checked using the electroencephalogram record obtained by measuring brain waves during sleep, as will be shown in Examples. Less sleepiness feeling on awakening, ease of falling asleep and the soundness of sleep, dreaming, the satisfaction of the length of sleep, and the feeling of fatigue can be evaluated using an OSA sleep inventory MA version (YAMAMOTO Yukari, TANAKA Hideki, TAKASE Miki, YAMAZAKI Katsuo, AZUMI Kazuo, and SHIRAKAWA Shuichiro: Standardization of revised version of OSA sleep inventory for middle age and aged, 10: 401-409, 1999), as will be shown in Examples. The feeling of fatigue can be evaluated using a Visual Analogue Scale (VAS) test sheet recommended by Japanese Society of Fatigue Science and shown in the guidelines on the method of evaluating the feeling of fatigue in anti-fatigue clinical evaluation of foods for specified health uses by the Japanese Society of Fatigue Science, as will be shown in Examples.

In the present invention, the phrase “to improve the quality of sleep” means that the quality of sleep is improved or raised. The improvement in the quality of sleep can be checked using, for example, at least one selected from the group consisting of a reduction in sleep onset latency, deep NREM sleep at the initial stage of sleep, an increase in the percentage of NREM sleep time in the total sleep time, an improvement in terms of less sleepiness feeling on awakening, an improvement in ease of falling asleep and soundness of sleep, an improvement in dreaming (for example, no frequent dreaming and no nightmare), an improvement in satisfaction with the length of sleep, and an increase in the feeling of recovery from fatigue (mitigation of the feeling of fatigue). The improvement in the quality of sleep can be checked using the conditions shown in Examples.

According to the sleep quality improving agent of the present invention, the quality of sleep can be effectively improved. Plants of the Fraxinus genus such as Fraxinus excelsior L. are conventionally used for nutritional enhancement and treatment of chronic rheumatism and podagra and used as herbal medicines and also as food in a specific region (the southeast of Morocco). There is a historical record that the original inhabitants of Eastern Russia ate Larix sibirica extract. Dihydroquercetin, dihydrokaempferol, and naringenin are components contained in Larix sibirica and are secondary metabolites of the plant. Therefore, the sleep quality improving agent of the present invention containing, as the effective ingredient, one or two or more selected from the group consisting of plants of the Fraxinus genus, extracts of the plants of the Fraxinus genus, Larix sibirica extract, dihydroquercetin, dihydrokaempferol, and naringenin is safe for the living body, and the user can use the sleep quality improving agent with a sense of security. In addition, the sleep quality improving agent of the present invention can reduce the time of nocturnal awakenings during sleep to maintain the time of sleep as will be shown in the Examples below, so that the effect on recovery from fatigue is more significantly exerted. Therefore, the sleep quality improving agent is expected to be applied to prevention and therapy of diseases related to a reduction in the quality of sleep such as diabetes, heart disease, hypertension, hyperlipidemia, and Alzheimer's disease. Accordingly, the sleep quality improving agent of the present invention is useful as final products such as food and drink products, pharmaceutical products, and quasi-pharmaceutical products.

No particular limitation is imposed on the subjects who ingest the sleep quality improving agent of the present invention or the sleep quality improving composition of the present invention. Examples of the subjects may include subjects who sleep poorly, subjects who are still sleepy on awakening, subjects who have a difficulty in falling asleep, subjects who cannot sleep soundly (who cannot sleep deeply), subjects who have a bad dream, subjects who feel fatigue, and subjects who feel fatigue even after sleep. In addition, subjects who do not have any particular problem can ingest the sleep quality improving agent on a daily basis for the purpose of improving or maintaining the quality of sleep.

Preferably, the sleep quality improving agent of the present invention is used as various food and drink products. Examples of the food and drink products may include beverages such as soft drinks, carbonated drinks, energy drinks, powdered drinks, fruit drinks, milk-based drinks, and jelly drinks, confectioneries such as cookies, cakes, chewing gums, candies, tablets, gummies, “manjyu (steamed buns with filing),” “yokan (adzuki bean jellies),” puddings, jellies, ice creams, and sherbets), processed marine products such as “kamaboko (boiled fish pastes),” “chikuwa (baked tubular rolls of fish pastes),” and “hanpenn (boiled cakes made of ground fish),”, processed livestock products such as hamburgers, hams, sausages, wiener sausages, cheeses, butters, yogurt, fresh creams, margarines, and fermented milk, soups such as powdered soups and liquid soups, staple foods such as rice, noodles (dried noodles, fresh noodles), breads, and cereals, and seasonings such as mayonnaise, shortening, dressing, sauce, “tare (dipping sauce),” and soy sauce.

The sleep quality improving agent of the present invention may be used as food and drink products such as health foods, functional foods, dietary supplements (supplements), foods for specified health uses, foods for medical use, foods for the sick, infant foods, foods for nursing care, and foods for the elderly. Of these, the sleep quality improving agent is preferably used as health foods and functional foods.

### EXAMPLES

The present invention will next be described by way of Examples.

### 1. Evaluation of Sleep Inducing Effect ((Example 1-1 and Comparative Example 1-1: Measurement of Amount of Activity of Mice)

To check the sleep inducing effect of an extract of a plant of the Fraxinus genus, the following test was performed using respective samples shown in TABLE 1.

The Fraxinus excelsior L. extract used (the sample in Example 1-1) was “fraxipure (trade name)” manufactured by NATUREX. A 0.5% by mass aqueous methyl cellulose solution (the sample in Comparative Example 1-1) was used as a control.

When a sample has the function of inducing sleep, the amount of activity always decreases after administration of the sample. Therefore, the sleep inducing effect of each sample was evaluated by determining whether or not the administration of the sample solution to a mouse caused a reduction in the amount of activity.

8-Week old or 9-week old male C57BL/6 mice were purchased from Japan SLC, Inc. and acclimatized for 3 to 6 days. The amount of activity during acclimatization was used to divide the mice into administration groups. Immediately before the onset of the dark period, Fraxinus excelsior L. extract was orally administered in an amount of 3 g/kg per body weight of a mouse (Example 1-1), or a 0.5% by mass aqueous methyl cellulose solution was orally administered in an amount of 10 mL/kg per body weight of a mouse (Comparative Example 1-1), and the amount of activity was measured for 24 hours after administration. Each administration group included 8 mice (n=8).

The amount of activity was measured under the following conditions. The mice were raised in individual cages, and an infrared camera was disposed above each cage. The imaging range of the infrared camera was divided into 64 sections, and the number of occurrences that the mouse crossed a section was measured. The number of occurrences was collected every 30 minutes.

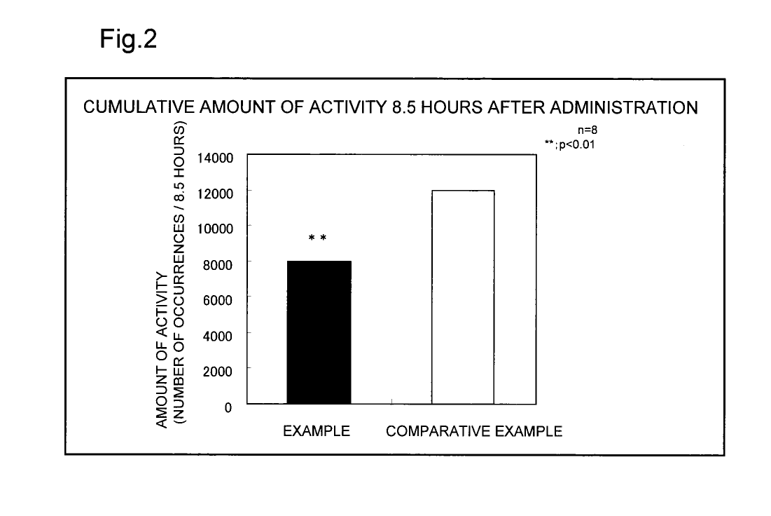

TABLE 1 shows the type of sample, and the cumulative amount of activity (the number of occurrences/8.5 hours) 8.5 hours after administration for each of Example 1-1 and Comparative Example 1-1. FIG. 2 shows the cumulative amount of activity of mice 8.5 hours after sample administration for each of the Example and Comparative Examples. In FIG. 2, “**” represents that a significant difference was found at p<0.01.

TABLE 1
(TYPE OF SAMPLE AND CUMULATIVE AMOUNT OF
ACTIVITY 8.5 HOURS AFTER ADMINISTRATION)

| | | COMPARATIVE |
|---|---|---|
| | | EXAMPLE 1-1 |
| | EXAMPLE 1-1 | CONTROL SOLUTION |
| | FRAXINUS | (0.5% BY MASS |
| | EXCELSIOR L. | AQUEOUS METHYL |
| | EXTRACT | CELLULOSE SOLUTION) |
| CUMULATIVE AMOUNT | 8000 | 12000 |
| OF ACTIVITY 8.5 | | |
| HOURS AFTER | | |
| ADMINISTRATION | | |
| (NUMBER OF | | |
| OCCURRENCES/8.5 | | |
| HOURS) | | |

The following can be seen from TABLE 1 and FIG. 2.

In Example 1-1 in which the Fraxinus excelsior L. extract was used as the sample, the cumulative amount of activity 8.5 hours after administration was significantly lower than that in Comparative Example 1-1 in which the control solution was administered.

These results show that the Fraxinus excelsior L. extract has a sleep inducing effect.

### 2. Evaluation of the Effect of Improving the Quality of Sleep (Example 1-2 and Comparative Example 1-2: Measurement of Brain Waves of Mice)

To check the effect of the Fraxinus excelsior L. extract on the quality of sleep, the following experiment was performed to measure brain waves. More specifically, electrodes were attached to the head of a mouse, and the brain waves of the mouse that was allowed to move freely in a recording chamber were recorded to measure the time of “wake,” the time of “REM sleep,” and the time of “NREM sleep.”

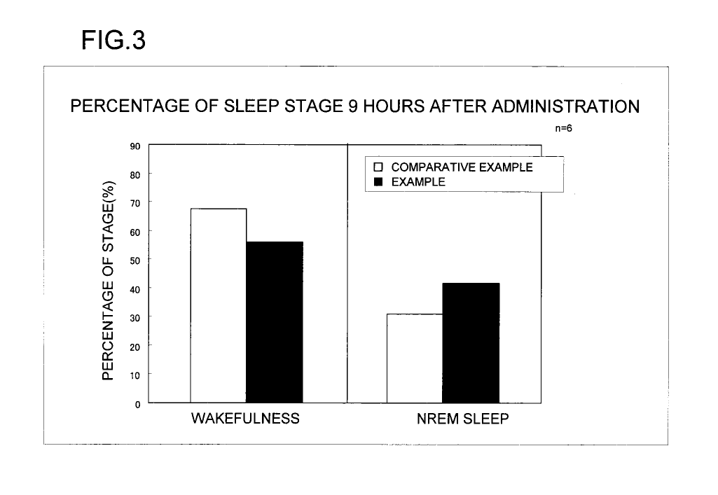

Electrodes for the brain waves and an electromyogram were attached to each of 8-week old male C57BL/6 mice purchased from Japan SLC, Inc. After the electrodes were attached, each mouse was recovered in a recovering chamber for 10 days. Then the mouse was transferred to the recording chamber, and cables were attached to the mouse. Brain wave analysis software SleepSign (registered trademark) Ver 3.0 (KISSEI COMTEC Co., Ltd.) was used to check whether or not the brain waves could be recognized, and the mouse was acclimatized for 3 days. Then the brain waves were measured for 24 hours to check whether or not a sleep-wakefulness rhythm was maintained, and the mice were divided into administration groups. Each administration group included 6 mice (n=6). Immediately before the onset of the dark period, a suspension prepared by suspending the Fraxinus excelsior L. extract in a 0.5% by mass aqueous methyl cellulose solution was orally administered in an amount of 3 g/kg in terms of the Fraxinus excelsior L. extract, or a control solution was orally administered in an amount of 10 mL/kg. Then the brain waves were recorded for 24 hours. The control solution was a 0.5% by mass aqueous methyl cellulose solution. After recording, the brain waves were automatically analyzed by the SleepSign (registered trademark) Ver 3.0, and the evaluator checked the results of the automatic analysis to divide the sleep into three sleep stages: a wakefulness stage, a REM sleep stage, and a NREM sleep stage. The ratio of the time of each sleep stage to the total brain wave measurement time (9 hours) was computed as a percentage and used as the percentage of the sleep stage 9 hours after administration. The average for 6 mice was computed (TABLE 2 and FIG. 3).

TABLE 2
(RESULTS OF BRAIN WAVE MEASUREMENT
(AVERAGE FOR 6 MICE))

| | | COMPARATIVE |
|---|---|---|
| | | EXAMPLE 1-2 |
| PERCENTAGE OF | EXAMPLE 1-2 | CONTROL SOLUTION |
| SLEEP STAGE 9 | FRAXINUS | (0.5% BY MASS |
| HOURS AFTER | EXCELSIOR L. | AQUEOUS METHYL |
| ADMINISTRATION (%) | EXTRACT | CELLULOSE SOLUTION) |
| WAKEFULNESS | 56.1(%) | 67.4(%) |
| NREM SLEEP | 41.6(%) | 31.1(%) |

The following can be seen from TABLE 2 and FIG. 3. In Example 1-2 in which the Fraxinus excelsior L. extract was used as the sample, the wakefulness time was reduced as compared to that in Comparative Example 1-2 in which the control solution was administered, and the NREM sleep was increased.

These results show that the quality of sleep is improved because the administration of the Fraxinus excelsior L. extract causes an increase in the percentage of NREM sleep time in the total sleep time. The results also show that nocturnal awakenings can be reduced and the sleep time can be maintained.

3. Evaluation of the Effect of Improving the Quality of Sleep (Example 1-3 and Comparative Example 1-3: Measurement of Human Brain Waves and Survey of Feelings about Sleep)

To check the effect of the Fraxinus excelsior L. extract on the quality of sleep of human, the following test was performed using respective samples shown in TABLE 3.

Tablets containing the Fraxinus excelsior L. extract (the content of the Fraxinus excelsior L. extract in two capsules: 500 mg) were prepared as the sample in Example 1-3. Placebo capsules containing no Fraxinus excelsior L. extract were prepared as the sample in Comparative Example 1-3. The details of these compositions are as shown in TABLE 3.

TABLE 3
(COMPOSITION OF TABLETS)

| COMPONENT (AMOUNT PER | | SAMPLE IN |
|---|---|---|
| SINGLE DOSE: AMOUNT OF | SAMPLE IN | COMPARATIVE |
| COMPONENT IN 2 CAPSULES) | EXAMPLE 1-3 | EXAMPLE 1-3 |
| FRAXINUS EXCELSIOR L. | 500 | 0 |
| EXTRACT (mg) | | |
| CRYSTALLINE CELLULOSE (mg) | 0 | 500 |
| TOTAL AMOUNT (mg) | 500 | 500 |

12 Adult male and female panelists with relatively bad sleep conditions were selected according to a survey performed in advance using the Pittsburgh Sleep Quality Questionnaire. Each of the 12 panelists was requested to orally ingest the sample in the above Example 1-3 one hour before going to bed, and a two-electrode type portable brain wave measurement device was attached to the panelist to measure the brain waves during sleep. Similarly, each of the same 12 panelists was requested to orally ingest the sample in Comparative Example 1-3 one hour before going to bed, and the two-electrode type portable brain wave measurement device was attached to the panelist to measure the brain waves during sleep. In the next morning, the panelists answered a questionnaire on feelings about sleep and a VAS test sheet for the feeling of fatigue. The number of times of administration of each sample and the number of measurements performed were four times for each panelist. The average sleep time of the panelists was 6.5 hours. The difference in sleep time between the panelist with the shortest sleep time and the panelist with the longest sleep time was 2 hours, and the difference was not significant.

### (Brain Wave Analysis)

The analysis of the brain waves was entrusted to SleepWell Co., Ltd. The time from the onset of sleep (the start of brain wave measurement) until appearance of NREM sleep was used as sleep onset latency. The depth of sleep can be determined by the power value of delta waves in the brain waves during sleep. The larger the power value of delta waves is, the larger the depth of sleep is. Immediately after the onset of sleep, NREM sleep appears, and then REM sleep appears. The power value of delta waves during NREM sleep immediately after the onset of sleep until appearance of REM sleep was used as the power value of delta waves at the initial stage of sleep.

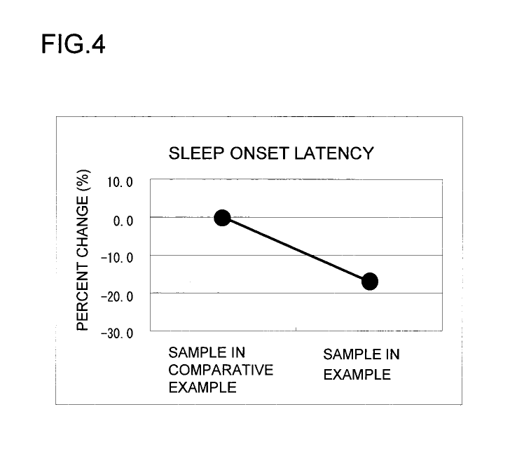

The values of sleep onset latency vary depending on individual panelists. Therefore, the sleep onset latency was measured four times for each panelist, and the average of the four measurements was computed. Then the percent change (%) in sleep onset latency relative to that when the placebo was ingested was computed for each panelist using the following formula (1-1) (TABLE 4 and FIG. 4).

Percent change (%) in sleep onset latency={(average sleep onset latency value after ingestion of Fraxinus excelsior L. extract)/(average sleep onset latency value after ingestion of placebo)}×100−100 [Formula (1-1)]

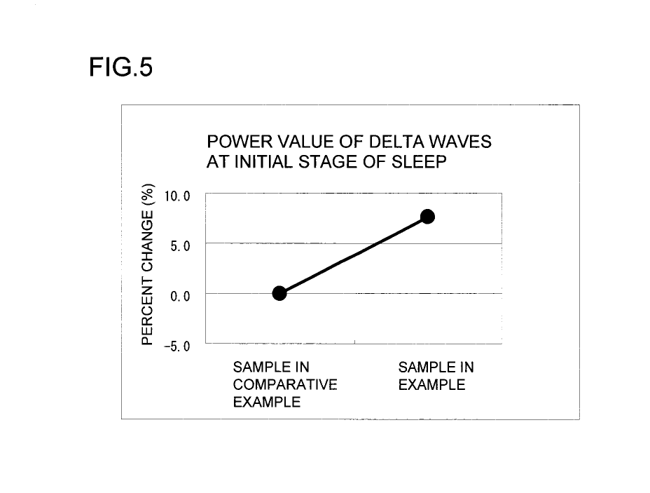

The power values of delta waves also vary depending on individual panelists. Therefore, the power value of delta waves was measured four times for each panelist, and the average of the four measurements was computed. Then the percent change (%) in the power value of delta waves relative to that when the placebo was ingested was computed for each panelist using the following formula (1-2) (TABLE 4 and FIG. 5).

Percent change (%) in power value of delta waves={(average power value of delta waves after ingestion of Fraxinus excelsior L. extract)/(average power value of delta waves after ingestion of placebo)}×100−100 [Formula (1-2)]

(Survey of Feelings about Sleep)

OSA sleep inventory MA version (YAMAMOTO Yukari, TANAKA Hideki, TAKASE Miki, YAMAZAKI Katsuo, AZUMI Kazuo, and SHIRAKAWA Shuichiro: Standardization of revised version of OSA sleep inventory for middle age and aged, Brain science and mental disorders, 10: 401-409, 1999) was used for a survey of feelings about sleep. The OSA sleep inventory MA version includes 20 questions and is an evaluation method for evaluating factor I: sleepiness feeling on awakening (i.e., less sleepiness feeling on awakening), factor II: initiation and maintenance of sleep (i.e., ease of falling asleep and an improvement in soundness of sleep), factor III: dreaming (i.e., having a good dream), factor IV: recovery from fatigue (i.e., lack of the feeling of fatigue), and factor v: sleep length (i.e., satisfaction with the length of sleep).

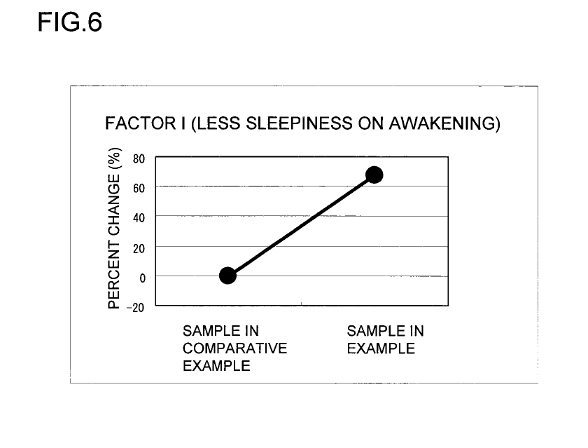

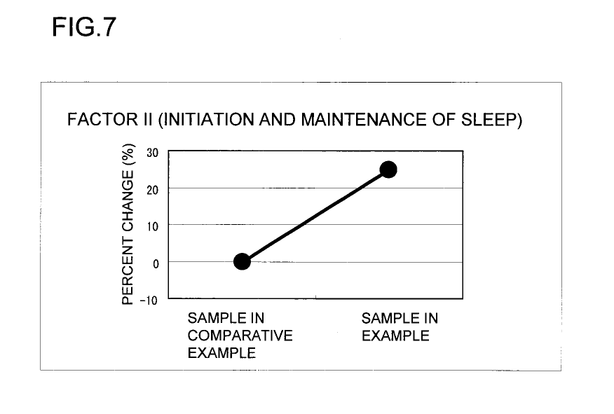

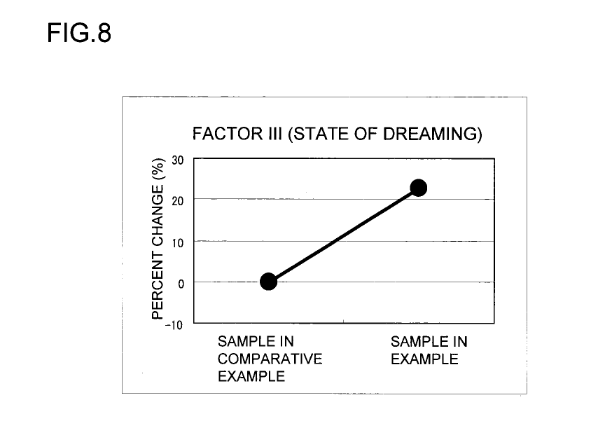

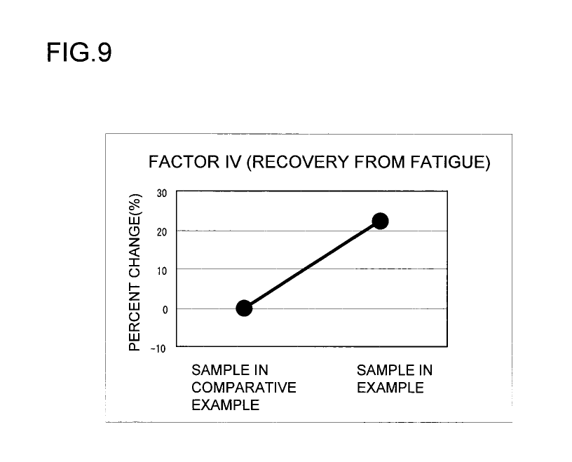

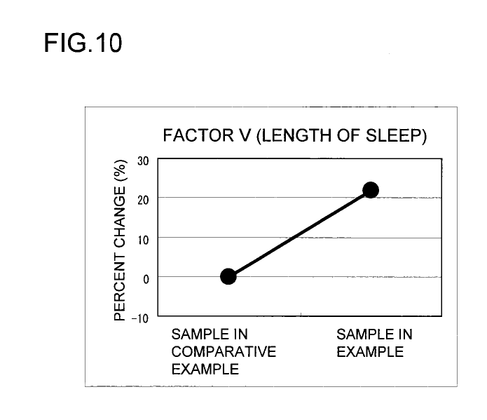

The scores of the respective factors were computed, and the average values of four measurements were computed for each panelist for each ingested sample. The percent change (%) in the score of each factor relative to that when the placebo was ingested was computed for each panelist using the following formula (1-3). The average percent change for the 12 panelists was computed (TABLE 5 and FIGS. 6 to 10).

Percent change (%) in score of each factor={(average value after ingestion of Fraxinus excelsior L. extract-containing yeast)/(average value after ingestion of placebo)}×100−100 [Formula (1-3)]

### (Survey on Feeling of Fatigue)

A Visual Analogue Scale (VAS) test sheet recommended by Japanese Society of Fatigue Science and shown in the guidelines on the method of evaluating the feeling of fatigue in anti-fatigue clinical evaluation of foods for specified health uses by the Japanese Society of Fatigue Science was used for the survey on the feeling of fatigue. More specifically, the left end of a horizontal straight line with a certain length was set to “the best feeling with no fatigue,” and the right end was set to “the worst feeling with exhaustion without motivation to do anything.” Each panelist marked a cross on the straight line as the answer to the feeling of fatigue on awakening in the morning with reference to the feeling shown at the left and right ends of the straight line.

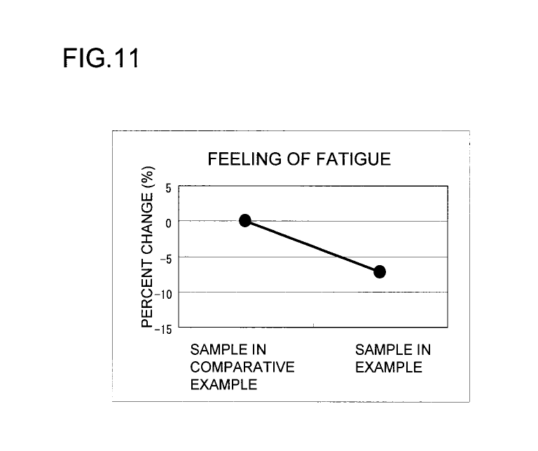

The distance from the left end of the straight line to the cross was measured to judge the feeling of fatigue. Specifically, it was judged that the smaller the value of the distance is, the less the feeling of fatigue is. The measurement was performed four times for each panelist for each ingested sample, and the average of the measurements was computed. The percent change (%) relative to placebo ingestion was computed for each panelist using the following formula (1-4). Then the average percent change for the 12 panelists was computed (TABLE 6 and FIG. 11).

Percent change (%) in feeling of fatigue={(average value after ingestion of Fraxinus excelsior L. extract)/(average value after ingestion of placebo)}×100−100 [Formula (1-4)]

### (Results of Brain Wave Analysis)

TABLE 4
RESULTS FOR SLEEP ONSET LATENCY AND NREM
SLEEP AT INITIAL STAGE OF SLEEP (AVERAGE
OF PERCENT CHANGES FOR 12 PANELISTS)

| | | SAMPLE IN |
|---|---|---|
| | SAMPLE IN | COMPARATIVE |
| | EXAMPLE 1-3 | EXAMPLE 1-3 |
| SLEEP ONSET LATENCY | −16.9% | 0% |
| POWER VALUE OF DELTA | 7.6% | 0% |
| WAVES AT INITIAL STAGE OF | | |
| SLEEP | | |

The following can be seen from TABLE 4 and FIGS. 4 and 5. In Example 1-3 in which the capsules of the Fraxinus excelsior L. extract were orally ingested as the sample, the sleep onset latency was shorter than that in Comparative Example 1-3 in which the placebo capsules were orally ingested. In addition, the power value of delta waves at the initial stage of sleep was large, and NREM sleep at the initial stage of sleep was sufficiently deep.

These results show that administration of the Fraxinus excelsior L. extract shortens sleep onset latency, deepens NREM sleep at the initial stage of sleep, and improves the quality of sleep effectively.

(Results of Survey of Feelings about Sleep)

TABLE 5
RESULTS OF SURVEY OF FEELINGS ABOUT SLEEP USING
OSA SLEEP INVENTORY MA VERSION (PERCENT CHANGE
IN EACH FACTOR (AVERAGE FOR 12 PANELISTS))

| | | SAMPLE IN |
|---|---|---|
| | SAMPLE IN | COMPARATIVE |
| | EXAMPLE 1-3 | EXAMPLE 1-3 |
| FACTOR I (LESS SLEEPINESS | 67.2% | 0% |
| FEELING ON AWAKENING) | | |
| FACTOR II (INITIATION AND | 24.9% | 0% |
| MAINTENANCE OF SLEEP) | | |
| FACTOR III (STATE OF | 22.7% | 0% |
| DREAMING) | | |
| FACTOR IV (RECOVERY FROM | 22.5% | 0% |
| FATIGUE) | | |
| FACTOR V (LENGTH OF SLEEP) | 21.9% | 0% |

The following can be seen from TABLE 5 and FIGS. 6 to 9. In Example 1-3 in which the capsules of the Fraxinus excelsior L. extract were orally ingested as the sample, the percent change in factor I (less sleepiness feeling on awakening) was higher than that in Comparative Example 1-3 in which the placebo capsules were orally ingested. The percent change in the score of factor II (initiation and maintenance of sleep), the percent change in factor III (state of dreaming), and the percent changes in factor IV (recovery from fatigue) and in factor V (length of sleep) were similar to the percent change in factor I. These results show that the effect of improving the quality of sleep was exerted by the administration of the Fraxinus excelsior L. extract to thereby improve sleepiness on awakening. These results also show that the effect of improving the quality of sleep was exerted to thereby improve ease of falling asleep and soundness of sleep. These results also show that the effect of improving the quality of sleep was exerted to thereby improve the state of dreaming. In addition, these results show that the effect of improving the quality of sleep was exerted to thereby increase the feeling of recovery from fatigue. In addition to the improvement of the quality of sleep, it can be seen that the lengthening effect of sleep time is exerted (satisfaction with the length of sleep is obtained).

### (Results of Survey on Feeling of Fatigue)

TABLE 6
RESULTS OF VAS TEST (PERCENT CHANGE IN FEELING
OF FATIGUE (AVERAGE FOR 12 PANELISTS))

| | | SAMPLE IN |
|---|---|---|
| | SAMPLE IN | COMPARATIVE |
| | EXAMPLE 1-3 | EXAMPLE 1-3 |
| FEELING OF | −7.2% | 0% |
| FATIGUE | | |

The following can be seen from TABLE 6 and FIG. 11. In Example 1-3 in which the capsules of the Fraxinus excelsior L. extract were orally ingested as the sample, a change to minus in comparison with Comparative Example 1-3 in which the placebo capsules were orally ingested was found, in sleepiness on awakening of the feeling of fatigue. These results show that the effect of improving the quality of sleep was exerted by the administration of the Fraxinus excelsior L. extract, so that the feeling of fatigue disappeared (the feeling of fatigue was mitigated).

Prescription Examples of the sleep quality improving agent of the present invention and the sleep quality improving composition of the present invention are shown below.

### Prescription Example 1-1

### Tablet (1-1)

10 mg of the Fraxinus excelsior L. extract, 60 mg of lactose, 80 mg of crystalline cellulose, 5 mg of carboxymethyl cellulose-Ca, and 5 mg of sucrose fatty acid ester were subjected to tablet compression in a conventional manner and produced tablets.

### Prescription Example 1-2

### Tablet (2-2)

250 mg of the Fraxinus excelsior L. extract, 40 mg of crystalline cellulose, 5 mg of carboxymethyl cellulose-Ca, and 5 mg of sucrose fatty acid ester were subjected to tablet compression in a conventional manner and produced tablets.

### Prescription Example 1-3

### Tablet (2-3)

Tablets were produced in a conventional manner using the following raw materials.

A granulated product (108 mg), the Fraxinus excelsior L. extract (20 mg), sorbitol (110 mg), partially pregelatinized modified starch (15 mg), magnesium phosphate (75 mg), calcium stearate (3 mg), menthol particles (40 mg), and aspartame (5 mg) were subjected to tablet compression in a conventional manner and produced tablets.

The above granulated product was produced by adding a 6% by mass aqueous hydroxypropyl cellulose solution (4,000 g) to erythritol (1,935 g) and cornstarch (300 g). The average particle diameter of the granulated product was 290 μm.

### Prescription Example 1-4

### Soft Capsules

Soft capsules were produced in a conventional manner using the following raw materials.

First, the following raw materials were mixed to prepare a filling: the Fraxinus excelsior L. extract (20 mg), vegetable oil (110 mg), glycerine fatty acid ester (10 mg), and beeswax (10 mg).

Soft capsules were produced using the filling and pork gelatin.

### 4. Evaluation of Sleep Inducing Effect ((Example 2-1, Example 2-2, Comparative Example 2-1, and Comparative Example 2-2: Measurement of Amount of Activity of Mice)

To check the sleep inducing effect of Larix sibirica extract and dihydroquercetin, the following test was performed using respective samples shown in TABLE 7 and TABLE 8.

“DIKVERTIN (trade name)” manufactured by FLAVIR was used as the Larix sibirica extract (the sample in Example 2-1). A reagent (MP Bio Japan K.K) was used for dihydroquercetin (the sample in Example 2-2). A 0.5% by mass aqueous methyl cellulose solution was used as a control (the samples in Comparative Examples 2-1 and 2-2).

When a sample has the function of inducing sleep, the amount of activity always decreases after administration of the sample. Therefore, the sleep inducing effect of each sample was evaluated by determining whether or not the administration of the sample solution to a mouse caused a reduction in the amount of activity.

8-Week old or 9-week old male C57BL/6 mice were purchased from Japan SLC, Inc. and acclimatized for 3 to 6 days. The amount of activity during acclimatization was used to divide the mice into administration groups. Immediately before the onset of the dark period, Larix sibirica extract was orally administered in an amount of 3 g/kg per body weight of a mouse, dihydroquercetin was orally administered in an amount of 2.7 g/kg per body weight of a mouse, or a 0.5% by mass aqueous methyl cellulose solution was orally administered in an amount of 10 mL/kg per body weight of a mouse, and the amount of activity was measured for 24 hours after administration. Each administration group included 8 mice (n=8) for Example 2-1 and Comparative Example 2-1, and 4 mice (n=4) for Example 2-2 and Comparative Example 2-2.

The amount of activity was measured under the following conditions. The mice were raised in individual cages, and an infrared camera was disposed above each cage. The imaging range of the infrared camera was divided into 64 sections, and the number of occurrences that the mouse crossed a section was measured. The number of occurrences was collected every 30 minutes.

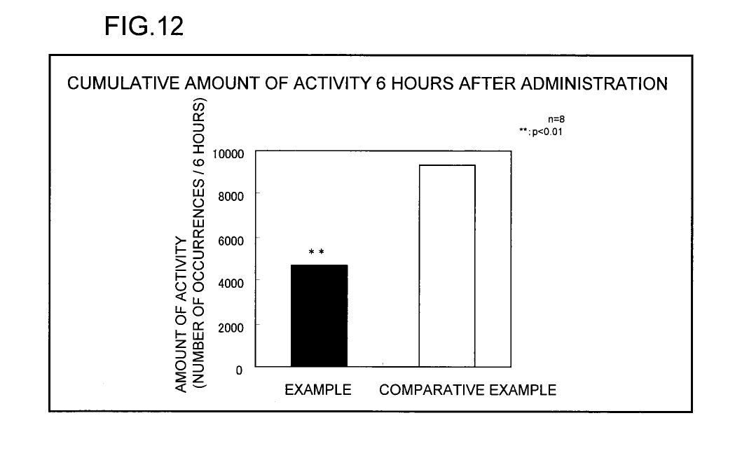

TABLE 7 shows the type of sample, and the cumulative amount of activity (the number of occurrences/6 hours) 6 hours after administration for each of Example 2-1 and Comparative Example 2-1. FIG. 12 shows the cumulative amount of activity of mice 6 hours after sample administration for each of the Example and Comparative Examples. In FIG. 12, “**” represents that a significant difference was found at p<0.01.

TABLE 7
(TYPE OF SAMPLE AND CUMULATIVE AMOUNT OF
ACTIVITY 6 HOURS AFTER ADMINISTRATION)

| | | COMPARATIVE |
|---|---|---|
| | | EXAMPLE 2-1 |
| | EXAMPLE 2-1 | CONTROL SOLUTION |
| | LARIX | (0.5% BY MASS |
| | SIBIRICA | AQUEOUS METHYL |
| | EXTRACT | CELLULOSE SOLUTION) |
| CUMULATIVE AMOUNT | 4700 | 9300 |
| OF ACTIVITY 6 | | |
| HOURS AFTER | | |
| ADMINISTRATION | | |
| (NUMBER OF OC- | | |
| CURRENCES/6 HOURS) | | |

The following can be seen from TABLE 7 and FIG. 12.

In Example 2-1 in which the Larix sibirica extract was used as the sample, the cumulative amount of activity 6 hours after administration was significantly lower than that in Comparative Example 2-1 in which the control solution was administered.

These results show that the Larix sibirica extract has a sleep inducing effect.

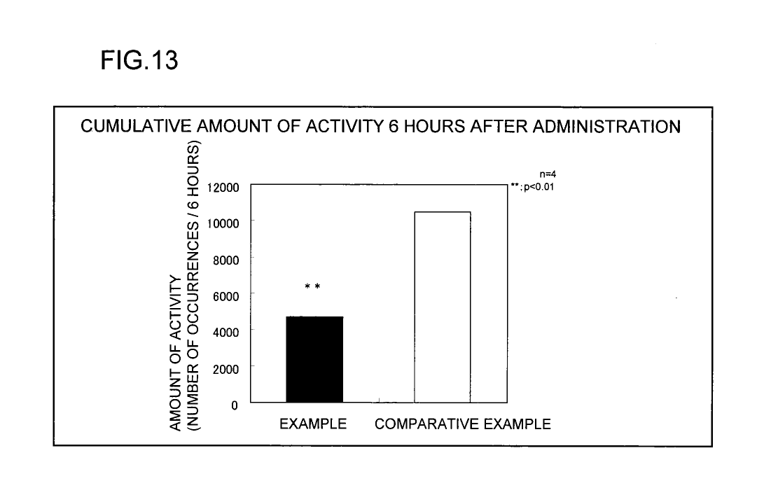

TABLE 8 shows the type of sample, and the cumulative amount of activity (the number of occurrences/6 hours) 6 hours after administration for each of Example 2-2 and Comparative Example 2-2. FIG. 13 shows the cumulative amount of activity of mice 6 hours after sample administration for each of the Example and Comparative Examples. In FIG. 13, “**” represents that a significant difference was found at p<0.01.

TABLE 8
(TYPE OF SAMPLE AND CUMULATIVE AMOUNT OF
ACTIVITY 6 HOURS AFTER ADMINISTRATION)

| | | COMPARATIVE |
|---|---|---|
| | | EXAMPLE 2-2 |
| | | CONTROL SOLUTION |
| | EXAMPLE 2-2 | (0.5% BY MASS |
| | DIHYDRO- | AQUEOUS METHYL |
| | QUERCETIN | CELLULOSE SOLUTION) |
| CUMULATIVE AMOUNT | 4700 | 10500 |
| OF ACTIVITY 6 HOURS | | |
| AFTER ADMINISTRA- | | |
| TION (NUMBER OF OC- | | |
| CURRENCES/6 HOURS) | | |

The following can be seen from TABLE 8 and FIG. 13.

In Example 2-2 in which dihydroquercetin was used as the sample, the cumulative amount of activity 6 hours after administration was significantly lower than that in Comparative Example 2-1 in which the control solution was administered.

These results show that dihydroquercetin has a sleep inducing effect.

5. Evaluation of the Effect of Improving the Quality of Sleep (Example 2-3 and Comparative Example 2-3: Survey of Feelings about Sleep)

To check the effect of the Larix sibirica extract on the quality of sleep of human, the following test was performed using respective samples shown in TABLE 9.

Tablets containing the Larix sibirica extract (the content of the Larix sibirica extract in six tablets: 60 mg) were prepared as the sample in Example 2-3. Placebo capsules containing no Larix sibirica extract were prepared as the sample in Comparative Example 2-3. The details of these compositions are as shown in TABLE 9.

TABLE 9
(COMPOSITION OF TABLETS)

| COMPONENT (AMOUNT PER | | SAMPLE IN |
|---|---|---|
| SINGLE DOSE: AMOUNT OF | SAMPLE IN | COMPARATIVE |
| COMPONENT IN 6 TABLETS) | EXAMPLE 2-3 | EXAMPLE 2-3 |
| LARIX SIBIRICA EXTRACT (mg) | 60 | 0 |
| CRYSTALLINE CELLULOSE (mg) | 1440 | 1500 |
| TOTAL AMOUNT (mg) | 1500 | 1500 |

12 Adult male and female panelists with relatively bad sleep conditions were selected according to a survey performed in advance using the Pittsburgh Sleep Quality Questionnaire. Each of the 12 panelists was requested to orally ingest the sample in the above Example 2-3 one hour before going to bed. Similarly, each of the same 12 panelists was requested to orally ingest the sample in Comparative Example 2-3 one hour before going to bed. In the next morning, the panelists answered a questionnaire on feelings about sleep and a VAS test sheet for the feeling of fatigue. The number of times of administration of each sample and the number of measurements performed were four times for each panelist. The average sleep time of the panelists was 6.5 hours. The difference in sleep time between the panelist with the shortest sleep time and the panelist with the longest sleep time was 2 hours, and the difference was not significant.

OSA sleep inventory MA version (YAMAMOTO Yukari, TANAKA Hideki, TAKASE Miki, YAMAZAKI Katsuo, AZUMI Kazuo, and SHIRAKAWA Shuichiro: Standardization of revised version of OSA sleep inventory for middle age and aged, Brain science and mental disorders, 10: 401-409, 1999) was used for a survey of feelings about sleep. The OSA sleep inventory MA version includes 20 questions and is an evaluation method for evaluating factor I: sleepiness feeling on awakening (i.e., less sleepiness feeling on awakening), factor II: initiation and maintenance of sleep (i.e., ease of falling asleep and an improvement in soundness of sleep), factor III: dreaming (i.e., having a good dream), factor IV: recovery from fatigue (i.e., lack of the feeling of fatigue), and factor v: sleep length (i.e., satisfaction with the length of sleep).

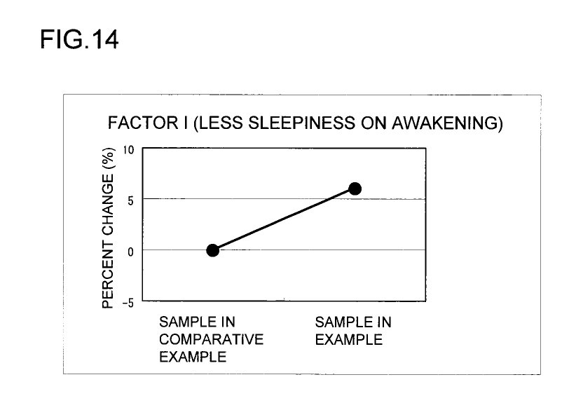

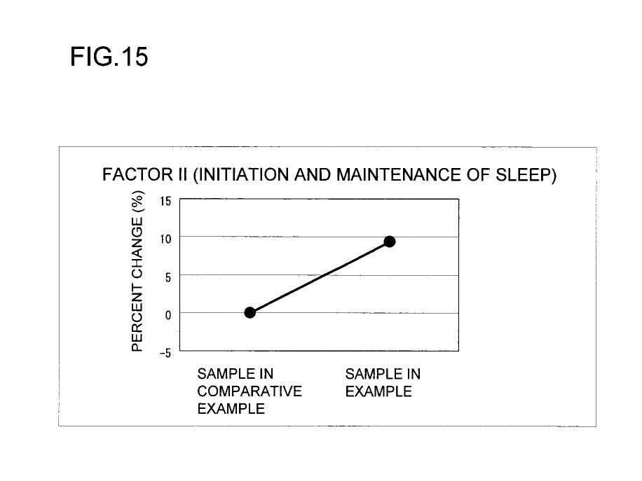

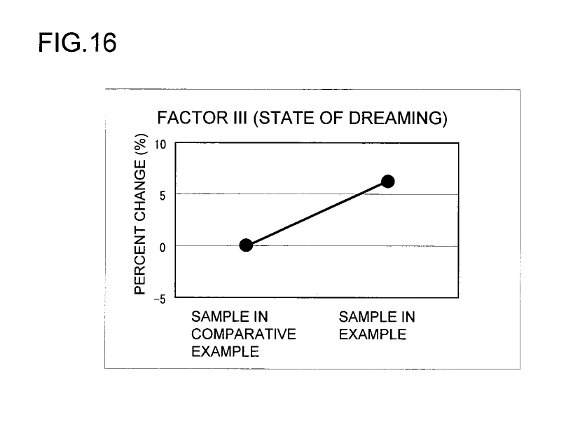

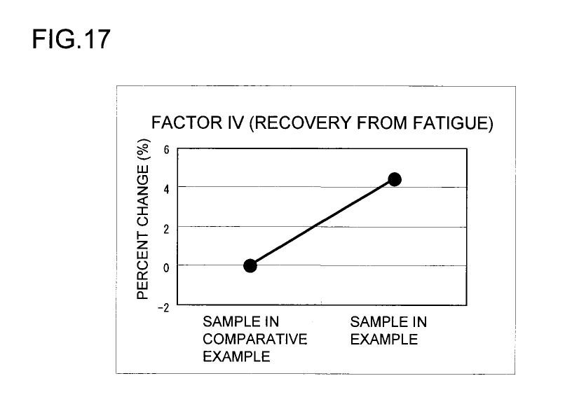

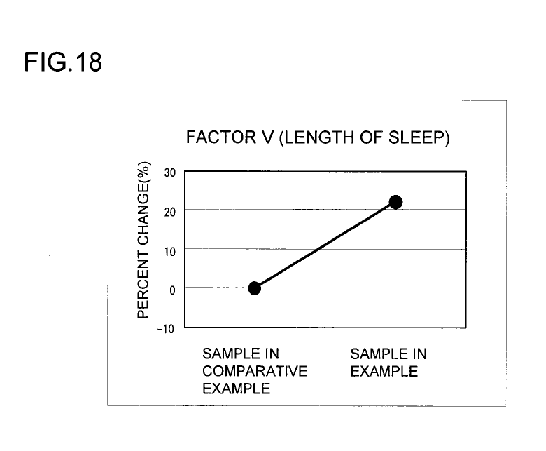

The scores of the respective factors were computed, and the average values of four measurements were computed for each panelist for each ingested sample. The percent change (%) in the score of each factor relative to that when the placebo was ingested was computed for each panelist using the following formula (2-1). The average percent change for the 12 panelists was computed (TABLE 10 and FIGS. 14 to 18).

Percent change (%) in score of each factor={(average value after ingestion of Larix sibirica extract-containing yeast)/(average value after ingestion of placebo)}×100−100 [Formula (2-1)]

TABLE 10
RESULTS OF SURVEY OF FEELINGS ABOUT SLEEP USING
OSA SLEEP INVENTORY MA VERSION (PERCENT CHANGE
IN EACH FACTOR (AVERAGE FOR 12 PANELISTS))

| | | SAMPLE IN |
|---|---|---|
| | SAMPLE IN | COMPARATIVE |
| | EXAMPLE 2-3 | EXAMPLE 2-3 |
| FACTOR I (LESS SLEEPINESS | 6.0% | 0% |
| FEELING ON AWAKENING) | | |
| FACTOR II (INITIATION AND | 9.3% | 0% |
| MAINTENANCE OF SLEEP) | | |
| FACTOR III (STATE OF | 6.2% | 0% |
| DREAMING) | | |
| FACTOR IV (RECOVERY FROM | 4.4% | 0% |
| FATIGUE) | | |
| FACTOR V (LENGTH OF SLEEP) | 22.1% | 0% |

The following can be seen from TABLE 10 and FIGS. 14 to 18. In Example 2-3 in which the capsules of the Larix sibirica extract were orally ingested as the sample, the percent change in factor I (less sleepiness feeling on awakening) was higher than that in Comparative Example 2-3 in which the placebo capsules were orally ingested. The percent change in the score of factor II (initiation and maintenance of sleep), the percent change in factor III (state of dreaming), and the percent changes in factor IV (recovery from fatigue) and in factor V (length of sleep) were similar to the percent change in factor I. These results show that the effect of improving the quality of sleep was exerted by the administration of the Larix sibirica extract to thereby improve sleepiness on awakening. These results also show that the effect of improving the quality of sleep was exerted to thereby improve ease of falling asleep and soundness of sleep. These results also show that the effect of improving the quality of sleep was exerted to thereby improve the state of dreaming. In addition, these results show that the effect of improving the quality of sleep was exerted to thereby increase the feeling of recovery from fatigue. In addition to the improvement of the quality of sleep, it can be seen that the lengthening effect of sleep time is exerted (satisfaction with the length of sleep is obtained).

Prescription Examples of the sleep quality improving agent of the present invention and the sleep quality improving composition of the present invention are shown below.

### Prescription Example 2-1

### Tablet (2-1)

10 mg of the Larix sibirica extract, 60 mg of lactose, 80 mg of crystalline cellulose, 5 mg of carboxymethyl cellulose-Ca, and 5 mg of sucrose fatty acid ester were subjected to tablet compression in a conventional manner and produced tablets.

### Prescription Example 2-2

### Tablet (2-2)

Tablets were produced in a conventional manner using the following raw materials.

A granulated product (108 mg), the Larix sibirica extract (20 mg), sorbitol (110 mg), partially pregelatinized modified starch (15 mg), magnesium phosphate (75 mg), calcium stearate (3 mg), menthol particles (40 mg), and aspartame (5 mg) were subjected to tablet compression in a conventional manner and produced tablets.

The above granulated product was produced by adding a 6% by mass aqueous hydroxypropyl cellulose solution (4,000 g) to erythritol (1,935 g) and cornstarch (300 g). The average particle diameter of the granulated product was 290 μm.

### Prescription Example 2-3

### Soft Capsules

Soft capsules were produced in a conventional manner using the following raw materials.

First, the following raw materials were mixed to prepare a filling: the Larix sibirica extract (20 mg), vegetable oil (110 mg), glycerine fatty acid ester (10 mg), and beeswax (10 mg).

Soft capsules were produced using the filling and pork gelatin.

1. An agent, comprising, as an effective ingredient, at least one selected from the group consisting of a plant of Fraxinus genus, an extract of the plant of Fraxinus genus, Larix sibirica extract, dihydroquercetin, dihydrokaempferol, and naringenin.

2. The agent according to claim 1, wherein the effective ingredient is a plant of the Fraxinus genus or an extract thereof.

3. The agent according to claim 1, wherein the plant of the Fraxinus genus is Fraxinus excelsior L.

4. The agent according to claim 1, wherein the extract of the plant of the Fraxinus genus is Fraxinus excelsior L. extract.

5. The agent according to claim 4, wherein the Fraxinus excelsior L. extract is an extract of seeds of Fraxinus excelsior L.

6. The agent according to claim 1, wherein the effective ingredient is Larix sibirica extract.

7. The agent according to claim 1, wherein the effective ingredient is at least one compound selected from the group consisting of dihydroquercetin, dihydrokaempferol, and naringenin.

8. The agent according to claim 1, wherein the agent shortens sleep onset latency.

9. The agent according to claim 1, wherein the agent deepens NREM sleep at an initial stage of sleep.

10-15. (canceled)

16. A composition, comprising the agent according to claim 1.

17. A method for improving quality of sleep, comprising administering the agent according to claim 1 to a person in need thereof.

18. The method according to claim 17, comprising administering Larix sibirica extract to the person in need thereof, as the effective ingredient.

19. The method according to claim 17, comprising administering at least one compound selected from the group consisting of dihydroquercetin, dihydrokaempferol, and naringenin to the person in need thereof, as the effective ingredient.

20. The method according to claim 17, comprising administering a plant of Fraxinus genus or an extract thereof to the person in need thereof, as the effective ingredient.

21. The method according to claim 20, wherein improvement of sleep comprises at least one selected from the group consisting of shortening sleep onset latency, deepening NREM sleep at an initial stage of sleep, increasing a percentage of NREM sleep time in a total sleep time, improving less sleepiness feeling on awakening, improving ease of falling asleep and soundless of sleep, improving dreaming, improving feeling of fatigue, and improving satisfaction with a length of sleep, with the plant of Fraxinus genus or the extract thereof.

22. The method according to claim 18, wherein improvement of sleep comprises at least one selected from the group consisting of shortening sleep onset latency, deepening NREM sleep at an initial stage of sleep, increasing a percentage of NREM sleep time in a total sleep time, improving less sleepiness feeling on awakening, improving ease of falling asleep and soundless of sleep, improving dreaming, improving feeling of fatigue, and improving satisfaction with a length of sleep, with the Larix sibirica extract.

23. The method according to claim 18, wherein improvement of sleep comprises at least one selected from the group consisting of shortening sleep onset latency, deepening NREM sleep at an initial stage of sleep, increasing a percentage of NREM sleep time in a total sleep time, improving less sleepiness feeling on awakening, improving ease of falling asleep and soundless of sleep, improving dreaming, improving feeling of fatigue, and improving satisfaction with a length of sleep, with the compound.
